(19) United States
(12) Patent Application Publication
(10) Pub. No.: US 2008/0154154 A1
(43) Pub. Date: Jun. 26, 2008

(54) INTERNET-BASED SYSTEM AND A METHOD FOR AUTOMATED ANALYSIS OF TACTILE IMAGING DATA AND DETECTION OF LESIONS

(75) Inventors: Armen P. Sarvazyan, Lambertville, NJ (US); Vladimir Egorov, Princeton, NJ (US); Sergiy Kanilo, Lawrenceville, NJ (US)

Correspondence Address: BORIS LESCHINSKY, P.O. BOX 72, WALDWICK, NJ, 07463, US

(73) Assignee: ARTANN LABORATORIES, INC., Lambertville, NJ (US)

(21) Appl. No.: 12/038,041

(22) Filed: Feb. 27, 2008

Related U.S. Application Data
(62) Division of application No. 10/866,487, filed on Jun. 12, 2004.
(60) Provisional application No. 60/478,028, filed on Jun. 13, 2003.

(51) Int. Cl. A61B 5/103; G06K 9/00

(52) U.S. Cl. 600/587; 382/128

(57) ABSTRACT

An Internet-based system is described including a number of patient terminals equipped with tactile imaging probes to allow conducting of breast examinations and collecting the 2-D digital data from the pressure arrays of the tactile imaging probes. The digital data is processed at the patient side including a step of detecting moving objects and discarding the rest of the data from further analysis. The data is then formatted into a standard form and transmitted over the Internet to the host system where it is accepted by one of several available servers. The host system includes a breast examination database and a knowledge database and is designed to further process, classify, and archive breast examination data. It also provides access to processed data from a number of physician terminals equipped with data visualization and diagnosis means. The physician terminal is adapted to present the breast examination data as a 3-D model and facilitates the comparison of the data with previous breast examination data as well as assists physicians in feature recognition and final diagnosis.

### CROSS REFERENCE DATA

This is a divisional application from a co-pending U.S. patent application Ser. No. 10/866,487 filed Jun. 12, 2004, which in turn claims the priority date benefit from a U.S. Provisional Application No. 60/478,028 filed Jun. 13, 2003 by the same inventors and entitled “Internet-based system for the automated analysis of tactile imaging data and detection of lesions”. Both of these applications are incorporated herein in their entirety by reference.

This invention was made with government support under SBIR Grants No. R43 CA91392 and No. R43/44 CA69175 awarded by the National Institutes of Health, National Cancer Institute. The government has certain rights in this invention.

### BACKGROUND OF THE INVENTION

1. Field of the Invention

The invention relates generally to a method and system for early detection of breast cancer using a home use hand-held tactile imaging device connected via Internet to the central database. Specifically, data collected on a regular basis, e.g. once a week, and sent via Internet to a central database will form a four-dimensional (3-D spatial data plus time data) representation that will be analyzed by a computer and a physician.

2. Discussion of Background

Breast cancer is the most common cancer among women in the United States, and is second only to lung cancer as a cause of cancer-related deaths. It is estimated that one in ten women will develop breast cancer during her lifetime. Benign lesions cause approximately 90 percent of all breast masses. A mass that is suspicious for breast cancer is usually solitary, discrete and hard. In some instances, it is fixed to the skin or the muscle. A suspicious mass is usually unilateral and non-tender. Sometimes, an area of thickening that is not a discrete mass may represent cancer.

Screening women 50 to 75 years of age significantly decreases the death rate from breast cancer. The most common tool for breast cancer screening is regular or digital mammography. Digitized images of breast can be stored and can be enhanced by modifying the brightness or contrast (e.g. as described in the U.S. Pat. No. 5,815,591). These images can be transmitted by telephone lines for remote consultation. Computer-aided diagnosis is applied to the digital images and is used to recognize abnormal areas found on mammogram (e.g. as disclosed in the U.S. Pat. Nos. 6,205,236; 6,198,838; and 6,173,034). It is important to note that 10 to 15 percent of all breast cancers are not detected by a mammogram. A palpable breast mass that is not seen on a mammogram should have a thorough diagnostic work-up including ultrasound and needle biopsy as well as close follow-up.

Ultrasonographic screening is useful to differentiate between solid and cystic breast masses when a palpable mass is not well seen on a mammogram. Ultrasonography is especially helpful in young women with dense breast tissue when a palpable mass is not visualized on a mammogram. Ultrasonography is not efficient for routine screening, primarily because microcalcifications are not visualized and the yield of carcinomas is negligible.

Palpatory self-examination, widely advised and taught to women as means of preclinical testing, contributes substantially to early cancer detection. Those women who bring the problem to their physicians, frequently themselves first detect a significant fraction of breast cancer. The major drawbacks of manual palpation include the necessity to develop special skills to perform self-examination, subjectivity and relatively low sensitivity. Women often do not feel comfortable and confident to make a decision whether there really are changes in the breast, and whether they should bring it to the attention of their doctors.

Earlier, self-palpation devices were developed (U.S. Pat. Nos. 5,833,633; 5,860,934; and 6,468,231 by Sarvazyan et al. incorporated herein in their entirety by reference) which utilized the same mechanical information as obtained by manual palpation conducted by a skilled physician. The disclosed earlier methods and devices provide for detection of tissue heterogeneity and hard inclusions by measuring changes in the surface stress pattern using a pressure sensor array applied to the tissue along with motion tracking data analysis.

Development of the Internet technology as a means of information transfer has laid the foundation for new fields of medicine such as telemedicine and telecare. With increasing accessibility of the Internet and other communication means, at-home monitoring of health conditions is now available to a much larger group of population. The home telecare system collects biomedical data, such as three-channel electrocardiogram and blood pressure, digitizes it and transmits over the long distance to a medical specialist. As the transmission technology becomes universally available, more cost effective and powerful wireless application of the telecare could be conceivable—remote monitoring of the general population for life threatening diseases. The set of vital biomedical and imaging data can be established to be continuously or periodically collected, transferred and maintained in a centralized medical database. Once received, patient data can be filtered through the automated data-mining and pattern recognition algorithms for the comprehensive analysis. If a meaningful change in patient records is detected by the system it will alarm her physician, so the patient could be invited to a clinic for further analysis and treatment.

A prior attempt at a remote health care solution for a limited set of conditions is described in the U.S. Pat. No. 4,712,562. A patient's blood pressure and heart rate are measured and the measurements are sent via telephone to a remote central computer for storage and analysis. Reports are generated for submission to a physician or the patient. U.S. Pat. No. 4,531,527 describes a similar system, wherein the receiving office unit automatically communicates with the physician under predetermined emergency circumstances.

U.S. Pat. No. 4,838,275 discloses a device for a patient to lay on or sit in having electronics to measure multiple parameters related to a patient's health. These parameters are electronically transmitted to a central surveillance and control office where an observer interacts with the patient. The observer conducts routine diagnostic sessions except when an emergency is noted or from a patient-initiated communication. The observer determines if a non-routine therapeutic response is required, and if so facilitates such a response.

Other prior attempts at a health care solution are typified by U.S. Pat. No. 5,012,411, which describes a portable self-contained apparatus for measuring, storing and transmitting detected physiological information to a remote location over a communication system. The information is then evaluated by a physician or other health professional.

U.S. Pat. No. 5,626,144 is directed to a system, which employs remote sensors to monitor the state of health of a patient. The patient is not only simply aware of the testing, but actively participates in the testing. The system includes a remote patient-operated air flow meter, which has a memory for recording, tagging, and storing a limited number of test results. The patient-operated air flow meter also has a display to allow the patient to view a series of normalized values, and provides a warning when the value falls below a prescribed percentage of a “personal best number” value as previously set by the patient himself. The patient-operated air flow meter also includes a modem for transmission of the tagged data over the telephone to a remote computer for downloading and storing in a corresponding database. The remote computer can be employed to analyze the data. This analysis can then be provided as a report to the health care provider and/or to the patient.

U.S. Pat. No. 6,263,330 provides a network system for storage of medical records. The records are stored in a database on a server. Each record includes two main parts, namely a collection of data elements containing information of medical nature for the certain individual, and a plurality of pointers providing addresses or remote locations where other medical data resides for that particular individual. Each record also includes a data element indicative of the basic type of medical data found at the location pointed to by a particular pointer. This arrangement permits a client workstation to download the record along with the set of pointers, which link the client to the remotely stored files. The identification of the basic type of information that each pointer points to allows the physician to select the ones of interest and thus avoid downloading massive amounts of data where only part of that data is needed at that particular time. In addition, this record structure allows statistical queries to be effected without the necessity of accessing the data behind the pointers. For instance, a query can be built based on keys, one of which is the type of data that a pointer points to. The query can thus be performed solely on the basis of the pointers and the remaining information held in the record.

Despite these and other advances of the prior art, there is still a need for a cost-effective and simple in use method and system for self-screening large number of women and provide for early warning of breast cancer or other abnormalities.

### SUMMARY OF THE INVENTION

It is the object this invention to overcome the disadvantages of the prior art and to provide a cost-effective system and method for mass population screening based on computerized diagnostic medical imaging using a home breast self-palpation device linked to a central database.

It is another object of the invention to provide such system and method in conjunction with advanced image enhancement algorithms and Internet-based data transfer for physician review and conclusions.

Another object of this invention is to provide an automated method and system for characterization of lesions using computer-extracted features from tactile images of the breast.

Another yet object of this invention is to provide an automated method and system for determination of spatial, temporal and hybrid features to assess the characteristics of the lesions in tactile images.

An additional object of this invention is to provide an automated method and system for classification of the inner breast structures from 3-D structural images and making a diagnosis and/or prognosis.

It is yet another object of the invention to provide a method and system for an enhanced 3-D visualization of breast tissue mechanical properties.

The above and other objects are achieved according to the present invention by providing a new and improved methods for the analysis of lesions in tactile images, including generating 3-D tactile images from 2-D tactile image data, and extracting features that characterize a lesion within the mechanical image data.

More specifically, an Internet-based system is described including a number of patient terminals equipped with tactile imaging probes to allow conducting of breast examinations and collecting the data from the pressure arrays of the tactile imaging probes. The data is processed at the patient side including a novel step of detecting moving objects and discarding the rest of the data from further analysis. The data is then formatted into a standard form and transmitted to the host system where it is accepted by one of several available servers. The host system includes a breast examination database and a knowledge database and is designed to further process, classify, and archive breast examination data. It also provides access to this data from physician terminals equipped with data visualization and diagnosis means. The physician terminal is adapted to present the breast examination data as a 3-D model and facilitates the comparison of the data with previous breast examination data as well as assists a physician in feature recognition and final diagnosis.

### BRIEF DESCRIPTION OF THE DRAWINGS

A more complete appreciation of the invention and many of the attendant advantages thereof will be readily obtained as the same becomes better understood by reference to the following detailed descriptions when considered in connection with the accompanying drawings, wherein:

FIG. 1 is a schematic diagram of the system for the automated analysis of lesions in tactile images according to the present invention.

FIG. 2 is a flow chart of tactile image enhancement procedure.

FIG. 3 illustrates tactile image enhancement and segmentation procedures.

FIG. 4 shows temporal sequence of segmented binary tactile images received in circular oscillation tissue examination mode.

FIG. 5 is a diagram of three-layer, feed-forward backpropagation network used as detection classifier.

FIG. 6 shows the detection ability of trained network shown in FIG. 5.

FIG. 7 is an example of tactile images for model structures.

FIG. 8 is a flow chart of the method for the automated analysis of lesions in tactile images based on direct translation of 2-D tactile images into a 3-D structure image.

FIG. 9 shows a flow chart illustrating another method for the automated analysis and characterization of lesions in tactile images based on substructure segmentation.

FIG. 10 shows a flow chart illustrating yet another method for the automated analysis and characterization of lesions in tactile images based on a 3-D model reconstruction.

FIG. 11 shows a flow chart illustrating yet another method for the automated analysis and characterization of lesions in tactile images based on sectioning 3-D model reconstruction, and finally

FIG. 12 is an example of a dynamic tactile image sequence of a malignant lesion.

### DESCRIPTION OF THE PREFERRED EMBODIMENTS OF THE INVENTION

Reference will now be made in greater detail to preferred embodiments of the invention, examples of which are illustrated in the accompanying drawings. Wherever possible, the same reference numerals will be used throughout the drawings and the description to refer to the same or like parts.

Advances in computer science and diagnostic technologies have revolutionized medical imaging providing physicians with the wealth of clinical data presented in the form of images. Images obtained as a result of an expensive and lengthy procedure often represent just an isolated frozen frame of a continuously changing picture. The majority of existing diagnostic techniques are based on deriving a statistical correlation between the recorded image, as a current representation of the state of the body and a disease. The relationship between the static medical image and a dynamic pathological process of a disease is indirect. While the advanced pathology will frequently result in visible changes that could be distinguished from the accepted standard, early diagnosis and monitoring could be achieved only by detection of minute temporal changes of clinically predefined normal state of an individual. Currently, medical images are just briefly examined by the attending physician and then are stored in the patient file. Significant diagnostic information hidden in these images may be missed if there is no data on temporal changes in the properties of the organ featured by the individual images. Modern digital data transfer and storage capabilities make possible incorporation of the fourth dimension, namely the time into the spatial medical representation leading to the 4-D imaging. In addition, a wealth of a new knowledge could be obtained if the 4-D images were integrated with the relevant information about the patient and stored in a centralized database. Computer-assisted analysis of such databases can provide a physician with comprehensive understanding of the etiology and dynamics of the disease, and can help him in decision-making process. Cross-referencing the 4-D image with the similar cases will tell physician “what to look for”. An immediate access to the integrated database will tell him “where to look” and will do it in a timely and cost efficient manner. Beyond 4-D image storage and retrieval, linking of the images and other information about the patient (such as a family history, history of the disease, complaints, symptoms, results of the tests presented in numerical form, patient's weight, height, age, gender, etc.) will allow physicians to perform complex rational searches through the entire image database.

In addition to the data mining, the constructed database will provide an open field opportunity for the development of unique diagnostically relevant pattern recognition. Finding patterns or repetitive characteristics within 4-D images for the patients with the similar symptoms will present the physician with the list of potential causes. It will provide the physician with new insights by suggesting reasons that might have been outside of the scope of intuitive diagnosis. Therefore creation of a centralized “smart” 4-D image database will not only help in physician's decision making but also improve its quality and accuracy.

The self-palpation device will provide a virtual interface between patient and physician for remote screening for breast cancer development through dynamic imaging of changes in mechanical properties of the breast tissue. Data collected on a regular basis, e.g. weekly or monthly, will be sent via Internet to the central database to form a four-dimensional (3-D plus time) image that will be analyzed by a computer and a physician. Monitoring of the image changes in time will enable the development of an “individual norm” for each patient. The deviation from this individual norm could indicate an emerging pathology.

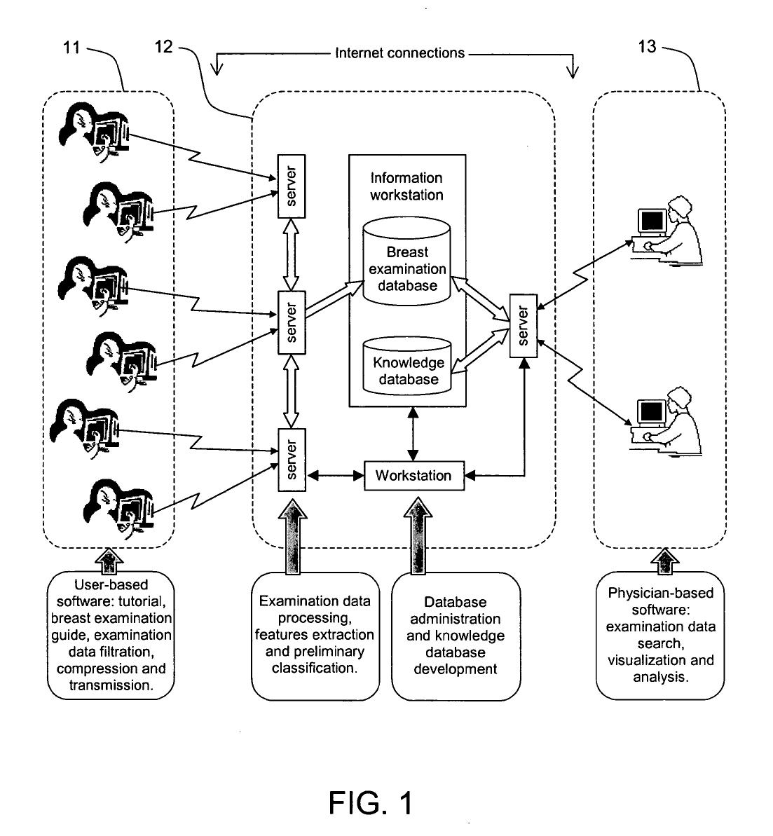

FIG. 1 shows a system block-diagram for implementing the method of automated analysis of tactile image data and detection of lesions in accordance with the present invention. A specialized host system (12) consisting of a number of patient and physician servers, an information database including a breast examination database and a knowledge database, and a workstation for administration and development. The breast examination database is connected to both patient and physician servers via communicating means to accept breast examination data from patients and notes from physicians. It is configured to process and store breast examination data, respond to service requests from the clients, and provide convenient access for both patients (11) and physicians (13) at any time. Patients provide data to the host system via patient terminals with patient communicating means (such as an Internet transmission means for example) preferably in a form of 2-D digital images acquired by pressure sensor arrays in tactile imaging probes described in detail elsewhere.

The host system includes a knowledge database configured analysis means for monitoring and automatically detecting temporal changes in breast properties based on historic data from the same patient as well as generally accepted norms. More specifically, the knowledge database is adapted to process stored breast examination data on the basis of biomechanical and clinical information, which includes established correlations between mechanical, anatomical, and histopathological properties of breast tissue as well as patient-specific data.

Breast examination data after being a subject of such preliminary evaluation as described above is then presented to physicians (13) at physician terminals. These terminals are equipped with additional communicating means and processing means for diagnostic evaluation of the breast examination data. These processing means are intended to facilitate a more comprehensive diagnosis and evaluation of data and assist physicians in a final diagnosis. Such processing means may include for example comprehensive image analysis, data searching means, comparison means to detect variations from prior examinations, etc. A physician is able to use either a Web browser or the client software to access the breast examination database and knowledge database, and communicate with the patients. The physician can enter his notes into the database, send recommendations to the patients, or seek advice from other specialists by sending the examination data for review, while keeping the patient personal information undisclosed. Participating physicians are provided with the preliminary diagnostic evaluation from the computerized data analysis of the accumulated relevant diagnostic data for the particular patient and the entire database. Physicians can conduct searches on the bulk of the accumulated data, find similar cases, and communicate with other physicians.

The data is distributed between a number of servers, configured according to the requirements for data storage and traffic intensity. As the data and traffic volume increase, new servers are added to keep up with the service expansion. After self-examination, the patient will submit data to the database using a client software equipped with an optional data privacy means for security and improved data consistency. Throughout the entire network, the patient is also provided with general information and technical support as well as the ability to participate in forums, read related articles, and receive instructions and training on using the breast self-palpation device. With the patient's history data stored in the database, the system delivers an unmatched capability of reviewing and investigating temporal changes in each case. The temporal visualization can be provided in the form of charts and animation displaying changes of important integral characteristics of the tissue and its distribution over time.

Data acquisition, transferring, processing and analyzing include the following general steps:
the client records on a patient computer the self-examination process during the acquisition phase;
during the following preliminary filtration analysis, the basic criteria for examination process quality, such as for example the presence of cancer and corresponding lesion parameters are calculated;
depending on the results of the preliminary analysis, the first set of recommendations are generated, such as for example to repeat the examination, transfer data to a global database, contact the physician, etc.;
the most representative data is sent to a global database. This can be done either in delayed mode reducing an overall system load or immediately in a more urgent case;
patient keeps trace (optionally) on her data processing through a dedicated web site. That site shows analysis status for the patient data;
data files from patients are directed via a web server to the virtual global database;
the server-based software conducts additional processing, classifies the data, and places the data to a substantial database server dedicated to this particular kind of data; and
the information from the virtual database is made accessible to physicians through special software, FTP and HTTP servers.

The main purpose of the physician's software is to prepare sophisticated inquiries to the virtual database. An inquiry incorporates an extensive set of breast cancer characteristics, which allow reducing the scope of a deliberate search. The parameters set increases when a new feature is derived from collected data and accepted by physicians.

Additional and optional features of the system of the invention are as follows:
Preliminary data filtration: a preliminary analysis can be conducted to reject sending an entire examination data stream or its parts if the data is of poor quality (too weak or saturated signals, high noise level, etc). In that case, the client software provides directions on what to do next: either repeat the examination or replace the device.
Automatic patient identification using hardware embedded features: the imaging probe device is intended for private use and, therefore, a serial number of the device automatically identifies the user. The Internet connection and data transferring can be done without the need to supply any additional identification information from the patient.
Software personalization: installed software and server-generated web-pages can use the user identification to make information more personal.
Suspended data uploading: it is not necessary to send examination data immediately after the examination is over, the client computer installed software (or device) can accumulate data in its own long-term memory and send the data at a more convenient or scheduled time.
Automatic result checking: there is no need to check the web site periodically for results of examination analysis, the software periodically checks for availability of such results and sends audible or visual message to the patient indicating its availability.

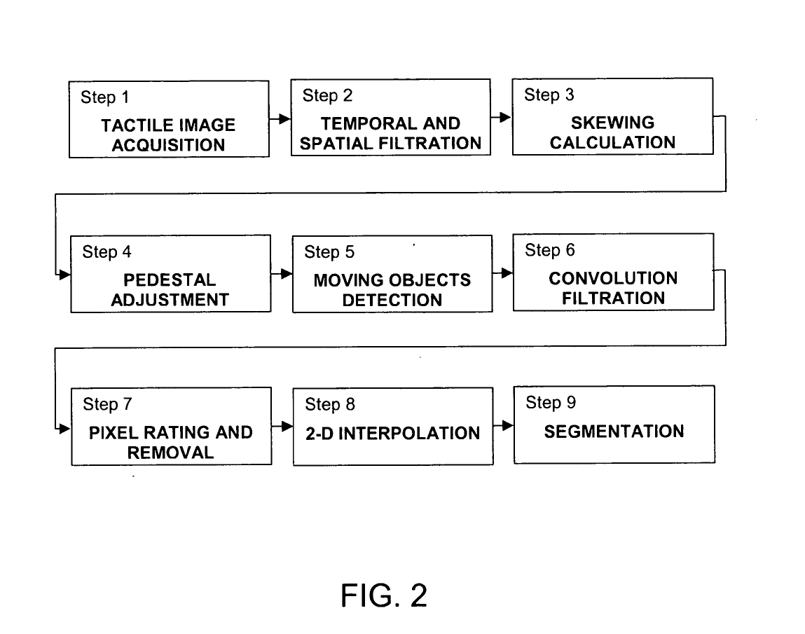

FIGS. 2, 3 and 4 illustrate tactile image enhancement and segmentation procedures to prepare data for input layer of the convolution network. This preparation is designed to minimize the data transmission to the network at a later point and includes the following steps:

### Step 1—tactile image acquisition;

### Step 2—temporal and spatial filtration;

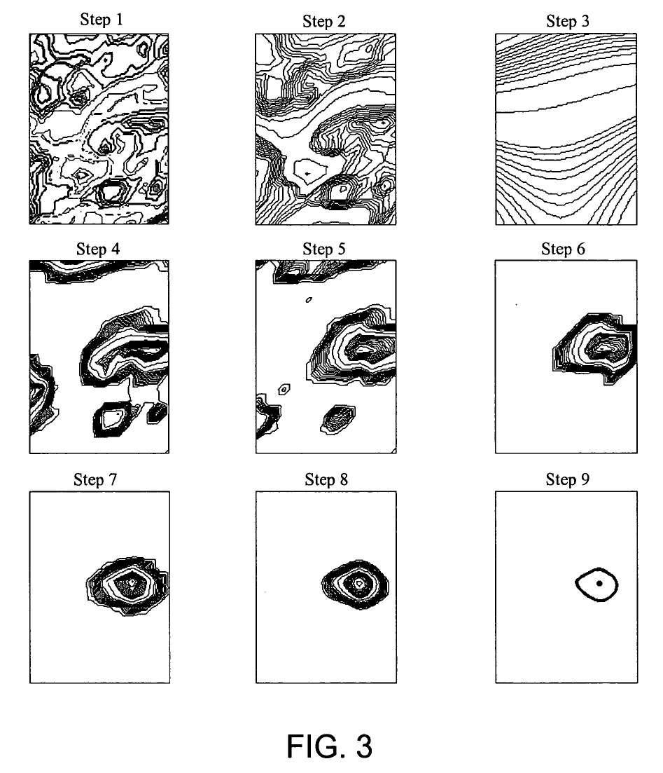

Step 3—skewing calculation. Skewing calculation consists of determination of a base surface supported by tactile signals from periphery sensors. This surface (base) is shown in step 3 on FIG. 3. Image shown in step 3 is subtracted from the image shown in the step 2 and the result is shown in step 4;

### Step 4—pedestal adjustment;

Step 5—moving objects detection. Step 5 is the most important step in this sequence. In this step, a prehistory for each tactile sensor is analyzed to find a signal minimum within about ½ to 1 second, which is then subtracted from the current image to detect moving structure objects in underlying tissue. All other information is discarded. This step allows a substantial reduction in data transmitted for further analysis as all information pertaining to non-moving objects is selectively removed from further processing;
Step 6—convolution filtration. In step 6, a weight factor for each tactile sensor signal is calculated in accordance with its neighborhood. Data from other sensors having the weight factor below a predetermined threshold is removed;

### Step 7—pixel rating and removal; A 2-D convolution of the image from step 6 and finite impulse response filter are both computed in this step;

Step 8—2-D interpolation. Step 8 comprises a bicubic surface interpolation where the value of an interpolated point is a combination of the values of the sixteen closest points, and finally
Step 9—segmentation. Step 9 is the edge and center detection to transform a tactile image shown in step 8 into a segmented binary image. Edge points can be calculated using image convolution with edge-detected matrix (for example 5 by 5 pixels). Center point may be a center mass point inside closed contour or just a maximum point in the image.

Importantly, steps 2-4 may be considered as preliminary processing steps, while steps 6-9 are final data processing steps to fit the data in a standard format for further transmission to the network.

An additional optional step is to provide a feedback signal indicating that the examination was done satisfactorily and sufficient data was collected for further analysis.

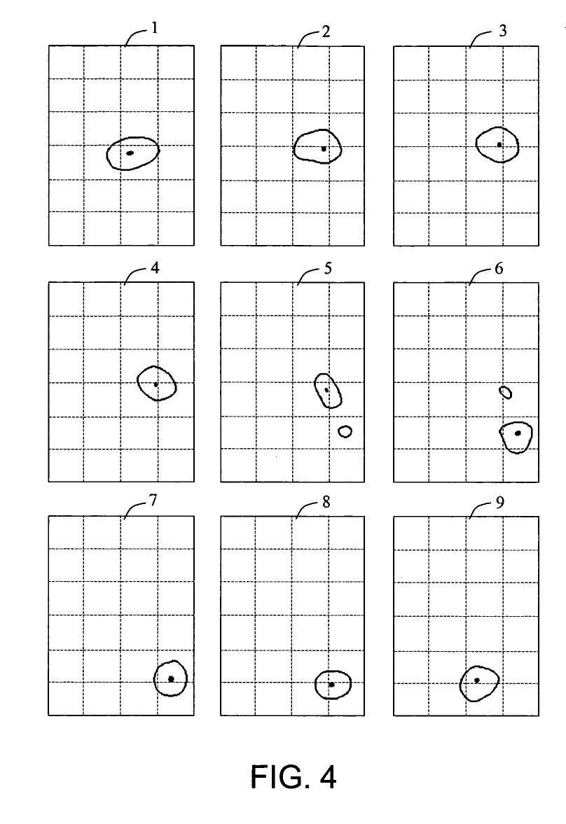

FIG. 4 shows temporal sequence of segmented binary tactile images received in tissue examination mode of circular oscillation. Closed contour corresponds to a lesion. This image sequence is then supplied to an input of a convolution network as described below in more detail.

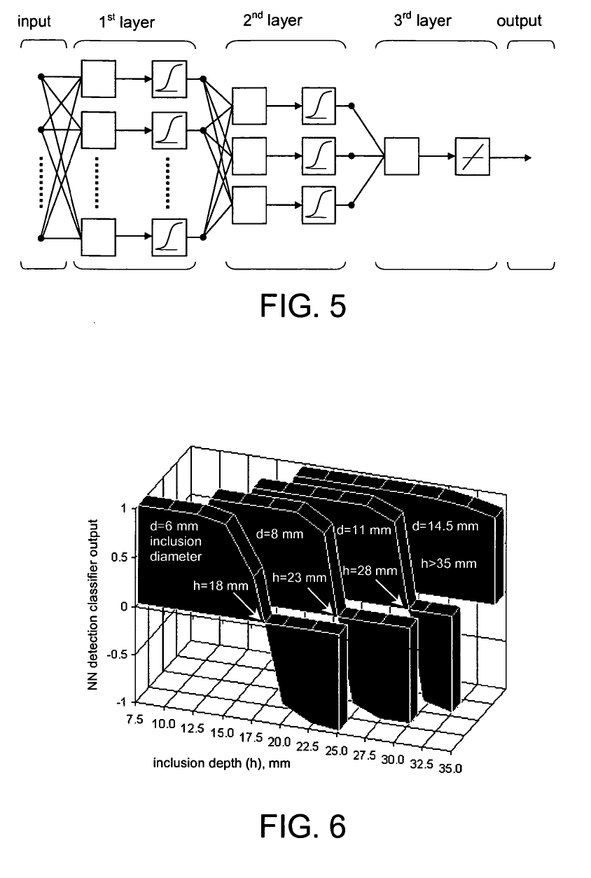

Simple and fast neural networks can be advantageously used for automated lesion detection. FIG. 5 shows a three-layer, feed-forward network including 10 input neurons in the first layer, 3 neurons in the second layer, and 1 in the third (output) layer. There is a connection present from each neuron to all the neurons in the previous layer, and each connection has a weight factor associated with it. Each neuron has a bias shift. The backpropagation algorithm guides the network's training. It holds the network's structure constant and modifies the weight factors and biases. The network was trained on 90 kernels, 65 of which contained lesions of different size and depth, and 25 kernels had no lesion.

FIG. 6 shows the example of a detection ability of such trained network for lesions having different sizes and depths. The set of features was comprised of average pressure, pressure STD, average trajectory step, trajectory step STD, maximum pressure, maximum pressure STD, size of a signal surface, signal surface STD, average signal, and extracted signal STD. Arrows show the detectability thresholds for inclusions of different diameter as a function of depth.

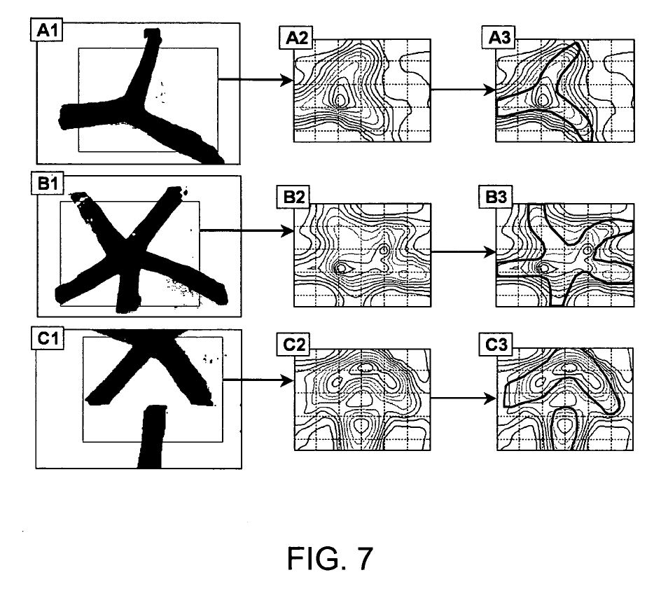

FIG. 7 shows sample tactile images (A2, B2, C2) of a model three-point star (A1), a five-point star (B1), and their combination (C1). The quality of such tactile images may be sufficient not only for detecting tissue abnormality but also for differentiating lesions based on their characteristic geometrical features. Quite probably, tactile imaging under certain conditions might allow for differentiating of different types of breast lesions such as fibrocystic alteration, cyst, intraductal papilloma, fibroadenoma, ductal carcinoma, invasive and infiltrating ductal carcinoma. A neural network self-organizing feature construction system could be advantageously used for this purpose. The basic principle in the system is to define a set of generic local primary features, which are assumed to contain pertinent information of the objects, and then to use unsupervised learning techniques for building higher-order features from the primary features as well as reducing the number of degrees of freedom in the data. In that case, final supervised classifiers will have a reasonably small number of free parameters and thus require only a small amount of pre-classified training samples. The feature of extraction is also envisioned where the classification system can be composed of a pipelined block structure, in which the number of neurons and connections decrease and the connections become more adaptive in higher layers.

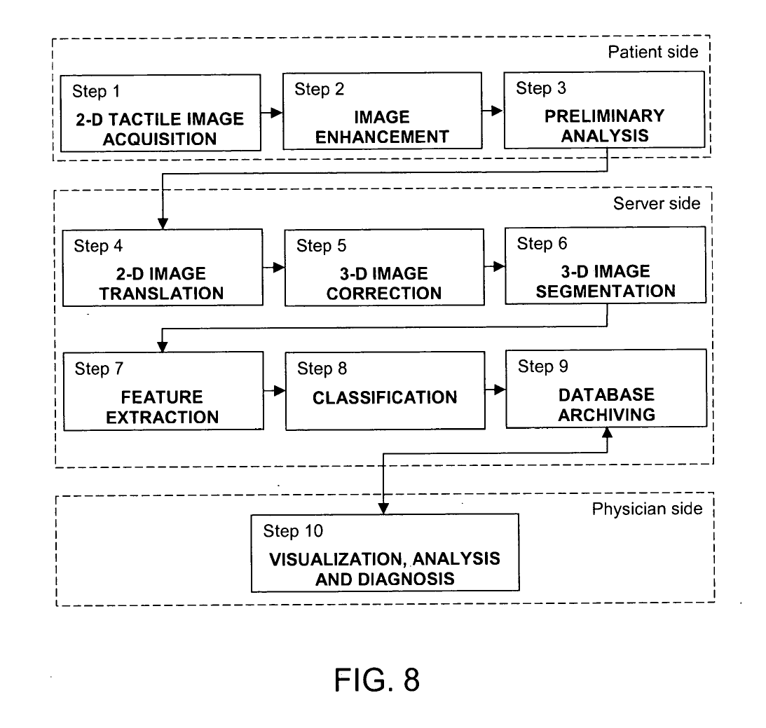

FIG. 8 shows a flow chart illustrating a first automated method for the analysis and characterization of lesions contained in tactile images according to the present invention. As shown on FIG. 8, the initial acquisition of a set of mechanical images comprising a presentation of the 2-D images in digital format is performed in real time during breast self-examination (step 1). Image enhancement (step 2) and preliminary data analysis (step 3) are fulfilled on patient side to prepare preliminary breast examination data before transmitting it to the server side of the host server network. The image analysis at the server side consists of the following consecutive steps:
translation of each image using image recognition technique from the 2-D image into a 3-D structural image (step 4), where as the third coordinate (Z-coordinate) is accordingly the coordinate from the tactile sensor array positioning data or average tactile pressure or another integral/hybrid parameter from those listed above;
3-D image correction by means of convolution of newly-incorporated 2-D tactile data with existing 3-D neighborhood (step 5);
image segmentation to identify the regions of interest of the breast and lesions (step 6);
spatial, temporal, and/or hybrid feature extraction (step 7);
rule-based, analytic, and/or artificial neural network classification (step 8);
archiving of processed breast examination data into a database (step 9); and
analysis by a physician of the breast examination data (step 10).

Visualization of data can be based on volume rendering, surface rendering, wire framing, slice or contour representation, and/or voxel modifications. In the segmentation process (step 6, FIG. 8), a detection process consists of three steps: segmentation of the 3-D image, localization of possible lesions, and segmentation of these possible lesions.

The purpose of segmenting the breast region from the tactile images is twofold:
to obtain a volume of interest which will require scanning in future to monitor the temporal changes of lesions; and
to produce a more detailed processing and rendering to visualize the location and shape of detected lesions with respect to a certain anatomical landmark such as a nipple.

The aim of lesion localization is to obtain points in the breast corresponding to a high likelihood of malignancy. These points are presumably part of a lesion. Lesion segmentation aims to extract all voxels that correspond to the lesion. Lesion detection is either performed manually, using an interactive drawing tool, or automatically by isolating voxels that have a rate of pressure uptake higher than a pre-defined threshold value.

Lesion segmentation can be performed by image processing techniques based on local thresholding, region growing (2-D), and/or volume growing (3-D). After detection, the feature extraction stage is employed (step 7). This stage consists of three components: extraction of temporal features, extraction of spatial features, and extraction of hybrid features. Features are mathematical properties of a set of voxel values that could reflect by themselves an underlying pathological structure. Many known methods can be used for this purpose, such as for example a directional analysis of the gradients computed in the lesion, and/or within its isosurface, and quantifying how the lesion extends along radial lines from a point in the center.

After the feature extraction stage, the various features are merged into an estimate of a lesion in the classification stage (step 8). Artificial neural networks, analytic classifiers as well as rule-based methods can be applied for this purpose. The output from a neural network or other classifiers can be used in making a diagnosis and/or prognosis. For example, with the analysis of the tactile 3-D images of the breast, the features can be used to either distinguish between malignant and benign lesions, or distinguish between the types of benign lesions, such as for example fibroadenoma, papilloma, or benign mastopathy.

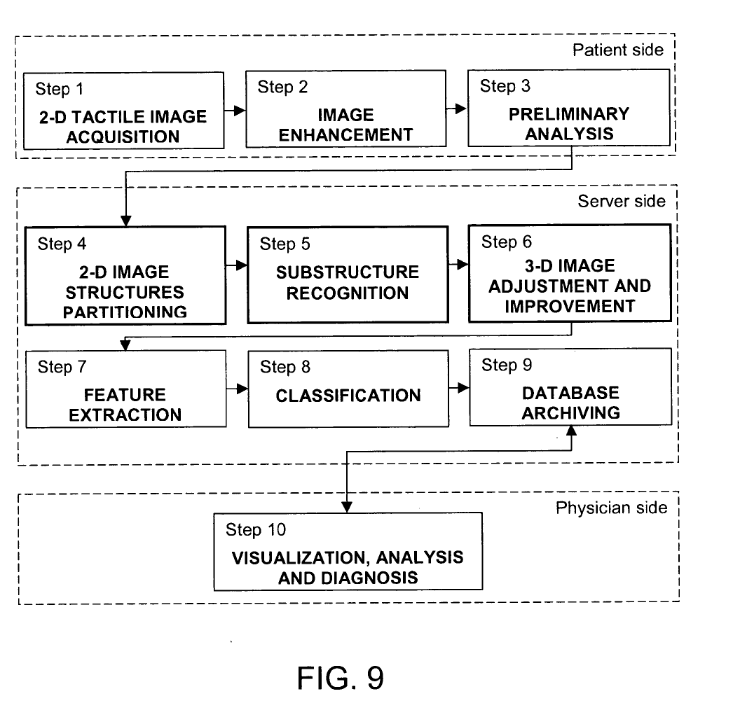

FIG. 9 shows a flow chart illustrating a second automated method of the invention based on substructure segmentation for the analysis and characterization of lesions in tactile images. The image analysis scheme at the server level consists of the following consecutive steps different from the first method described above:
2-D image structure partitioning (step 4);
deploying an image recognition technique for each substructure in the 2-D image to use new substructure information in a 3-D structure image (step 5);
3-D image adjustment and improvement after adding new substructure information (step 6);
spatial and/or temporal feature extraction (step 7);
rule-based, analytic, and/or artificial neural network classification (step 8), and
breast examination data archiving into a database (step 9).

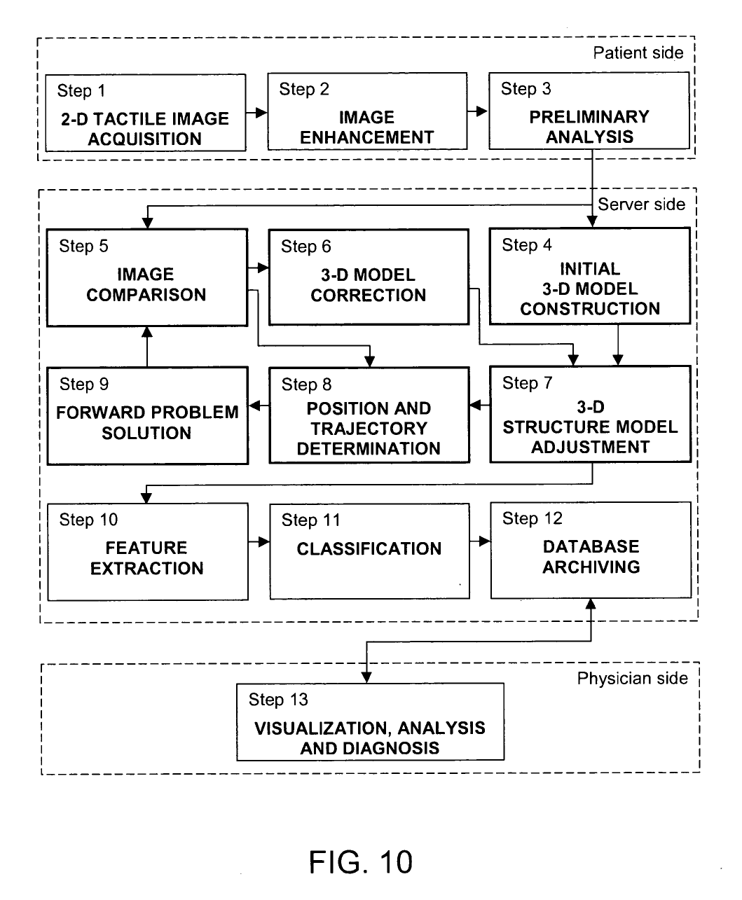

FIG. 10 shows a flow chart illustrating a third method based on a 3-D model reconstruction for the automated analysis and characterization of lesions in tactile images according to the present invention. Image analysis scheme at the server includes:
initial 3-D model construction (step 4);
cyclic optimization scheme (steps 5-9) including tactile sensor array position and trajectory determination with or without incorporated positioning system (step 8) for each analyzed frame,
forward problem solution (step 9),
2-D calculated and the 2-D analyzed images comparison (step 5);
3-D model correction (step 6).

As a result of this procedure, a 3-D structure model is formed with further feature extraction (step 10); classification (step 11); and database archiving (step 12).

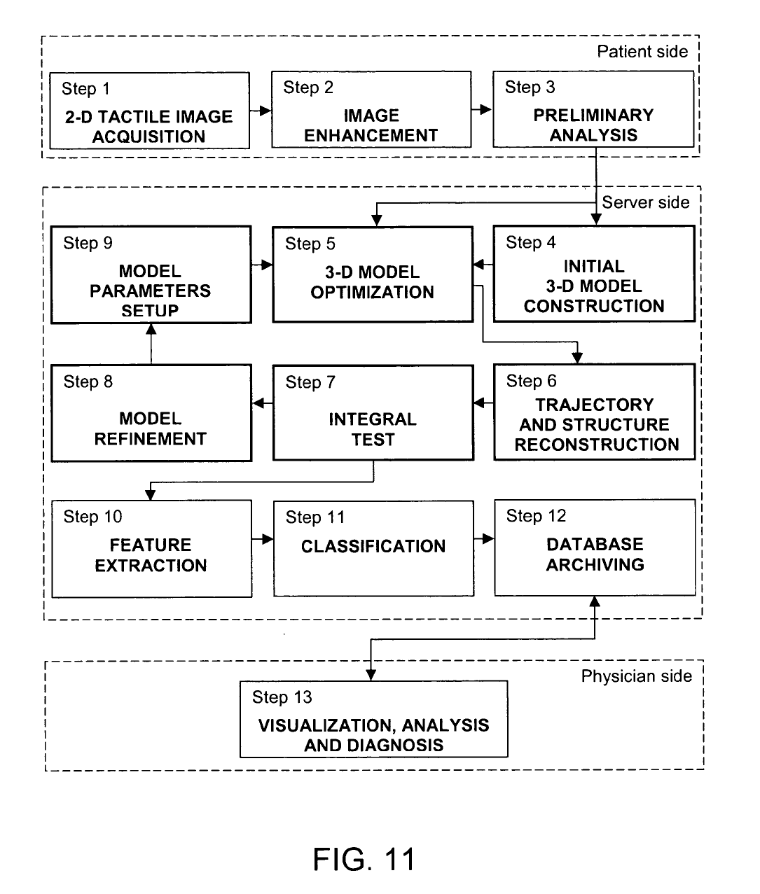

FIG. 11 shows a flow chart illustrating a fourth method for the automated analysis and characterization of lesions in tactile images according to the present invention. The image analysis scheme includes the steps of:
initial 3-D model construction (step 4);
solution of the least square problem enhanced with difference scheme (step 5),
trajectory and layer structure reconstruction (step 6),
integral test on overlapping tactile images (step 7);
interactive model refinement (step 8); and
setup for model approximation parameters and weight functions (step 9).

The model of an object is a multi-layer elastic structure. Each layer is defined as a mesh of cells with uniform elastic properties. From the static point of view, the pressure field on the working surface of a tactile imager is a weighed combination of responses from all layers. There is also an influence of pressing and inclination of pressure sensing surface. From the dynamics point of view, the layers shift and tactile image changes during the examination procedure. Assuming that the tactile sensor does not slip on the breast surface, the bottom layer can not be moved, and intermediate layers shift can be approximately linear, the equation for instant pressure image can be presented as follows:

$${W}_{p} P ( x , y , t ) = ( 1 + {α}_{x} {W}_{x} + {α}_{y} {W}_{y} + {α}_{z} {W}_{z} ) {\sum }_{i = 0}^{n} {W}_{i} {L}_{i} ( x + \frac{i}{n} dx , y + \frac{i}{n} dy , ϕ + \frac{i}{n} d ϕ , t )$$

where x, and y are coordinates tangential to the breast surface, z is a coordinate normal to surface, φ is an in-plane rotation angle, dx and dy are incline angles, t is time, P is resulting pressure field, Lᵢ is pressure distribution of i-layer, W is specified weight functions.

The layer approximation is much coarser than the source pressure images. Accordingly, the problem can be resolved with the least square algorithm. Differential representation of the pressure images sequence allows separation of the dynamic and static parameters and additional simplification of the problem. After solution of the problem and reconstruction of the trajectory of the tactile device and layers structure, the integral test is applied. It combines all data into a 3-D space and calculates integral residual between overlapping images. The analysis is over when residual becomes less than a prescribed threshold. Otherwise, a more detailed layer mesh is built and analysis the process is repeated. It is more advantageous in this case to start from a very coarse representation for the layers, because even several solutions for small grids can be processed faster than one problem with fine mesh. The resulting layer structure is visualized as a layer-by-layer or as a three-dimensional semi-transparent structure. The residuals also may be visualized, as they contain differential information, and in addition to integral layer picture they can reveal structural peculiarities of the breast under investigation.

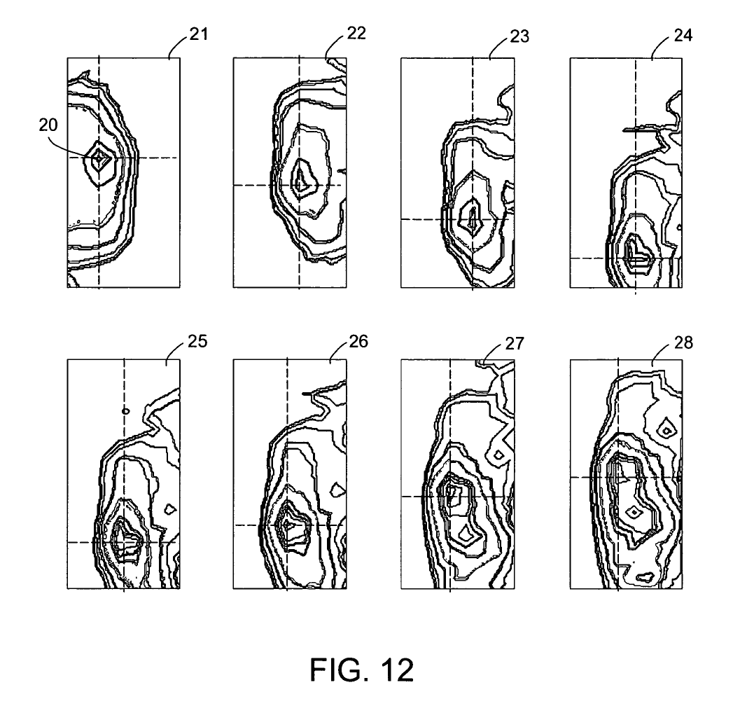

FIG. 12 is an illustration of step 1 of FIGS. 8-11 showing a real time tactile image sequence 21-28 revealing a lesion 20 using a tactile imaging device.

The 3-D tactile breast images can be transformed in a such way that it becomes suitable for visual and/or computerized comparison with images obtained from other modalities such as MR, mammography, and ultrasonography. The advantage of such comparison is to improve the performance of the diagnosis of breast cancer beyond the point of analysis of each individual modality alone. In addition, diagnosis by a physician may be facilitated when the tactile data is rendered similar to a visual appearance of a mammogram. For computerized analysis, rendering similar appearance is also desired to allow for an automated image comparison technique, such as registration by maximization of cross correlation.

Although the invention herein has been described with respect to particular embodiments, it is understood that these embodiments are merely illustrative of the principles and applications of the present invention. For example, despite the description in the preferred embodiment of the system for the characterization of lesions using computer-extracted features from tactile images of the breast, the methods of the present invention can be applied to characterization of other types of normal/abnormal anatomic regions. It is therefore to be understood that numerous modifications may be made to the illustrative embodiments and that other arrangements may be devised without departing from the spirit and scope of the present invention as defined by the appended claims.

What is claimed is:

1. A method for acquisition and analysis of tactile imaging data and detection of lesions in a soft tissue comprising the steps of:
a. providing a tactile imaging probe with an array of tactile sensors,
b. acquiring and preliminary processing tactile imaging data in a 2-D digital format using said imaging probe,
c. detecting moving objects data in said tactile imaging data,
d. retaining said moving objects data, while discarding other data,
e. digitally formatting said data and transmitting thereof to a network for further analysis and diagnosis.

2. The method as in claim 1, wherein said step of detecting said moving objects includes obtaining a prehistory for each of said tactile sensors within a predetermined period of time, determining a signal minimum within that period of time, and subtracting said minimum from the current level of signal to detect said moving objects in said underlying soft tissue.

3. The method as in claim 2, wherein said period of time is about ½ to 1 second.

4. The method as in claim 1, wherein said step “b” further including the steps of temporal and spatial filtration, skewing calculation, and pedestal adjustment.

5. The method as in claim 1, wherein said step “e” further including the steps of convolution filtration, pixel rating and removal, 2-D interpolation, and segmentation.
